(19) United States
(12) Patent Application Publication
(10) Pub. No.: US 2004/0259095 A1
(43) Pub. Date: Dec. 23, 2004

(54) Rapid high-throughput screen for detecting biological species in ballast water

(76) Inventors: Stephen C Cary, Lewes, DE (US); Kathryn j Coyne, Lewes, DE (US)

Correspondence Address: CONNOLLY BOVE LODGE & HUTZ, LLP, P O BOX 2207, WILMINGTON, DE, 19899, US

(21) Appl. No.: 10/493,995

(22) PCT Filed: Oct. 25, 2002
(86) PCT No.: PCT/US02/34123

(30) Foreign Application Priority Data
Oct. 30, 2001 (US) 60131335
Jul. 10, 2002 (US) 60394654

(51) Int. Cl. C12Q 1/68

(52) U.S. Cl. 435/6

(57) ABSTRACT

The present invention relates generally to compositions and methods to identify and enumerate invasive biological species present in ship ballast water. The present invention relates particularly, but not by way of limitation, to real-time, quantitative PCT methods.

### FIELD OF THE INVENTION

The present invention relates generally to methods and compositions to identify and enumerate invasive biological species present in ship ballast water. The present invention relates particularly, but not by way of limitation, to real-time, quantitative PCR methods as well as primers and probes for use therewith.

### BACKGROUND

Sea-faring vessels routinely dock at a first port, in a first part of the world, where it is loaded with a cargo that the vessel transports to a second port, typically in a second part of the world, where the cargo is unloaded. The vessel then returns to the first port where it is loaded with another cargo. Typically, the vessel travels empty from the second port back to the first port to pick up another cargo. The vessel is equipped with ballast tanks that can be filled with water to maintain the balance of the vessel on an even keel when it travels without a cargo. Conventional ballast tanks can include valves usually mounted over apertures through tank bulkheads. The valves are actuated to move water between and into and out of various ballast tanks to trim the vessel when empty of cargo or when carrying an unevenly distributed cargo.

The vessel fills its ballast tanks by taking on sea water, usually at its cargo discharge port. The sea water is charged into the ballast tanks at the same time that the vessel off loads its cargo. The vessel then travels to its cargo loading port where it takes on cargo while at the same time it empties at least some and typically all of its ballast tanks by discharging the ballast water into the loading port water environment.

The ballast water intake is below the water line of a vessel usually at or near the vessel hull bottom. Harmful water-borne organisms associated with ballast water contamination include, for example, algae, zooplankton, zebra mussels and other organisms that are indigenous to the cargo discharge port. Significant quantities of these indigenous organisms are loaded into the ballast tanks along with the water. The vessel then transports these organisms to a cargo loading port where the organisms are discharged into the water environment along with discharged ballast water. Typically these organisms are not indigenous to the water environment of the cargo loading port. Some of these organisms may be deleterious to and very much unwanted in the loading port environment.

Examples of harmful, water-borne organisms include Pfiesteria complex species, heterotrophic and mixotrophic dinoflagellates recognized as harmful algal bloom species. Harmful algal bloom (HAB) is the phenomenon also known as “red tide” and “toxic tide.” It is believed the worldwide frequency and distribution of HAB species is increasing. Of the approximately 5,000 known species of marine phytoplankton, about 300 can occur in sufficient concentration to discolor water, and at least 90 are classified as HAB species because they express potent toxins that adversely affect the health of human and fish populations. Other HAB species, while not directly harmful to humans, damage fish populations thereby damaging local economies. Toxicity-associated Pfiesteria species, including P. piscicida and P. shumwayae, have had adverse effects on the health and economies of the Chesapeake Bay (Maryland) and the Albermarle-Pamlico Sound (North Carolina) estuaries.

Harmful algal bloom is not unique to Atlantic waters. The Pacific Ocean hosts HAB species including the toxic dinoflagellates Gymnodinium catenatum and Alexandrium tamarense that are associated with paralytic shellfish poisoning. The detection of three species of toxic dinoflagellate cysts in ships' ballast waters led in 1990 to the introduction in Australia of special ballast water quarantine regulations that are now being adopted on an international basis by 132 countries.

The zebra mussel (Dreissena polymorpha) is another example of an unwanted organism that has been spread by ballast water. The zebra mussel was first found in the mid eighteenth century in the northern Caspian Sea and in the Ural River. Since then the mussel has quickly spread to other parts of the world by means of ballast water discharge. The mussel has threadlike tentacles that enable the mussel to adhere to any vertical or horizontal surface. The mussel reproduces quickly and attaches to any hard underwater surface. It is particularly adherent to the shell of another mussel. Stacks of mussels can completely clog water intake orifices. In a short time, they reach population densities in excess of 30,000 mussels per square meter. Zebra mussels have been known to completely shut down municipal water treatment plants and industrial water systems.

The spread of the zebra mussel and other nonindigenous organisms is an immense problem. The mussel was found in the Great Lakes in late 1988. It is believed that it was carried to North America in the ballast of ships from Europe. It was first prevalent in Lake Erie. Since then, the mussel has spread into Lake Michigan and into rivers of the Midwest and Northeast.

In 1996, Congress passed the National Invasive Species Act (P. L. 104-332) to stem the spread of nonindigenous organisms by ballast water discharge. The act reauthorized the Great Lakes ballast management program and expanded applicability to vessels with ballast tanks. The Act requires the Secretary of Transportation to develop national guidelines to prevent the spread of organisms and their introduction into U.S. waters via ballast water of commercial vessels. The Act establishes guidelines that require that vessels that enter U.S. waters after operating undertake ballast exchange in the high seas. In this method, a vessel empties its ballast on the high seas and refills the ballast tanks with sea water. However, the emptying of ballast tanks causes an imbalance that makes the exchange of ballast water exchange on the high seas both dangerous and sometimes impossible because of weather conditions. Additionally, high seas exchange requires manpower that many vessels do not have or cannot economically provide. There remains the need for a effective and economical ballast water test methods to prevent the spread of non-indigenous organisms.

Environmental monitoring programs to identify harmful organisms are in place in many areas, for example, The Environmental Protection Agency has promulgated the National Harmful Algal Bloom Research and Monitoring Strategy which strategy having an initial focus on pfiesteria species. The present invention may form a partial response to need defined in the national strategy.

Other objects and advantages will become apparent from the following disclosure.

### SUMMARY OF INVENTION

The present invention provides methods and compositions for detecting the presence of invasive biological species in the ballast water of a ship. According to an aspect of the present invention methods, compositions (e.g., primers and probes), and kits are provided to enumerate the biological species found in ballast water.

According to an aspect of the present invention, a method is provided to identify water-borne organisms associated with harmful algal bloom, the method comprises the steps of: (a) providing an aliquot of water, possibly containing biological organisms, (b) fractionating said aliquot by size to yield a plurality of fractions, (c) isolating DNA from at least one of said fractions, (d) amplifying said DNA through the process of high throughput, real-time PCR, wherein said PCR comprises at least one forward genus- specific primer and at least one reverse genus-specific primer to yield an amplified DNA amplicon, (e) contacting said amplicon with at least one species-specific labeled probe to form an amplicon/labeled probe complex; and (f) detecting (and optionally quantitating) said amplicon/labeled probe complex by observing the presence of - or measuring the amount of -said amplicon/labeled probe complex by means of monitoring said labeled probe.

According to an aspect of the present invention, a method is provided to identify water borne organisms associated with harmful algal bloom, wherein said organisms are transported in the ballast of a ship.

According to an aspect of the present invention, a method is provided to identify dinoflagellates. According to a further aspect of the present invention, a method is provided to identify dinoflagellates selected from the group consisting of Pfiesteria, Gymnodinium, Chattonella, Alexandrium, and Aureococcus.

According to an aspect of the present invention, a method is provided to identify invasive organisms transported in the ballast of a ship, wherein said organism is a zebra mussel (Dreissena).

According to an aspect of the present invention, a method to identify and enumerate water-borne biological organisms is provided. The method comprising the steps of: (a) providing an aliquot of water, possibly containing biological organisms; (b) fractionating the aliquot of water using at least one filter having a size appropriate to the targeted organism; (c) isolating DNA from said fractions; (d) amplifying the isolated DNA using the process of high throughput, real-time PCR, wherein the isolated DNA is amplified using genus-specific nucleic acid primers; and (e) detecting the amplified DNA using species-specific labeled probes.

According to an aspect of the present invention, the aliquot of water is obtained from the ballast of a ship. According to a further aspect of the present invention, the aliquot of water is obtained from an environmental sampling.

According to an aspect of the present invention, the biological organisms are targeted with respect to their size. Such targeting may comprise serial filtration. Serial filtration comprises the steps of (a) filtering an aliquot of water, using at least a first filter having a first pore size, thereby yielding a first retentate and a first filtrate; and (b) filtering the filtrate through at least a second filter, having a second pore size smaller than the first pore size, yielding a second retentate and a second filtrate. The retentate is defined as the material that is trapped on and in the filter. The filtrate is defined as the liquid volume that passes the filter and including any solid matter present in that liquid volume. For the purposes of the instant disclosure the term “pore” is understood to include, and to be interchangeable with, the term “mesh.“

According to an aspect of the present invention, template DNA is selected according to the size of the organism from which said DNA derives.

According to an aspect of the present invention, means are provided for the specific amplification and quantification of predetermined DNA. According to a further aspect of the invention, specificity of amplification is provided by appropriate selection of primers used to control the polymerase chain reaction (PCR) used to amplify the DNA of interest. According to a further aspect of the invention, specificity of detection is provided by appropriate selection of probes used to detect the amplified DNA. Persons skilled in the art will understand that primers are characteristic sequences of DNA found within the DNA sequence of the target organism.

According to an aspect of the present invention, primers suitable for the identification of harmful, water-borne organisms are provided. According to a further aspect the present invention provides primers comprising sequences specified by SEQ ID NO.: N, where N is an integer in the bounded interval 1 to 12, inclusive. In other words, SEQ ID NO: 1 through SEQ ID NO: 12, all inclusive.

According to an aspect of the present invention, probes suitable for the quantification of harmful, water-borne organisms are provided. According to a further aspect the present invention provides probes comprising sequences specified by SEQ ID NO.: O, where O is an integer in the bounded interval 13 to 16, inclusive.

According to a further aspect of the present invention, at least one kit is provided suitable for the identification and enumeration of water-borne species. Generally, the reagents and devices described herein are packaged to include many if not all of the necessary components for performing the reactions described herein. For example, the kits may include any of templates, polymerases, primers, probes, buffers and other chemical agents, nucleotides, sample materials, control materials, devices, or the like. Such kits also typically include appropriate instructions for using the devices and/or reagents, and in cases where reagents are not predisposed in the devices themselves, with appropriate instructions for introducing the reagents into the devices.

According to an aspect of the present invention, a method is provided to identify harmful water-borne organisms associated with ballast water contamination, or non-indigenous organisms spread by ballast water discharge, the method comprising the steps of: (1) providing an aliquot of water, possibly containing biological organisms; (2) fractionating said aliquot by size to yield a plurality of fractions; (3) isolating DNA from at least one of said fractions; (4) amplifying said DNA through the process of PCR, wherein said PCR comprises at least one forward genus-specific primer and at least one reverse genus-specific primer to yield an amplified DNA amplicon; (5) contacting said amplicon with at least one species-specific labeled probe; and (6) detecting said amplicon.

According to an aspect of the present invention, a method is provided to identify waterborne organisms associated with harmful algal bloom, wherein said organisms are dinoflagellates selected from the group consisting of Pfiesteria, Gymnodinium, Chattonella, AZexandrium, and Aureococcus.

According to an aspect of the present invention, a method is provided to identify waterborne organisms associated with harmful algal bloom, wherein said organisms are transported in the ballast of a ship.

According to an aspect of the present invention, a method is provided to identify waterborne organisms associated with harmful algal bloom, wherein said fractionating comprises serial filtration.

According to an aspect of the present invention, a method is provided to identify waterborne organisms transported in the ballast of a ship.

According to an aspect of the present invention, a method is provided to identify invasive organisms transported in the ballast of a ship, wherein said organism is a zebra mussel (Dreissena).

According to an aspect of the present invention, a method is provided to identify waterborne biological organisms, wherein said serial filtration comprises the steps of: (a) filtering said aliquot through at least a first filtration means having a first pore size, thereby obtaining a first filtrate and a first retentate; and (b) filtering said first filtrate through at least a second filtration means wherein said second filtration means has a second pore size smaller than said first pore size, thereby obtaining at least a second filtrate and a second retentate.

According to an aspect of the present invention, a method is provided to identify water borne biological organisms, wherein said aliquot comprises ship ballast water.

According to an aspect of the present invention, a method is provided to identify water borne biological organisms, wherein said aliquot comprises an environmental sampling.

According to an aspect of the present invention, a method is provided to identify water-borne biological organisms, wherein isolating DNA comprises the steps of: (a) extracting said retentate with an organic solvent thereby forming an aqueous fraction and an organic fraction; and (b) extracting DNA from said aqueous fraction thereby forming template DNA.

According to an aspect of the present invention, a method is provided to identify water borne biological organisms, wherein amplifying said DNA employs at least one forward primer and at least one reverse primer.

According to an aspect of the present invention, a method is provided to identify waterborne biological organisms, wherein high throughput real-time PCR analysis comprises the steps of: (a) providing an automated system for detection of amplified DNA sequences; (b) providing a charged plate compatible with said system, wherein said plate comprises a plurality of wells; (c) providing said extracted DNA to said charged plate; (d) amplifying said DNA; and (e) detecting said amplified DNA.

According to an aspect of the present invention, a method is provided to identify waterborne organisms associated with harmful algal bloom, wherein said automated DNA detection system comprises operating instructions.

According to an aspect of the present invention, a method is provided to identify water borne biological organisms, wherein/said plate comprises: at least one well established as a negative control; at least one well established as a positive control; at least one well provided test DNA; a plurality of wells containing sample DNA; appropriate primers; and appropriate probes.

According to an aspect of the present invention, a method is provided to identify waterborne biological organisms, wherein detecting said DNA amplicon comprises hybridizing said PCR-amplified DNA amplicon with a labeled probe.

According to an aspect of the present invention, a microarray hybridization method is provided.

According to an aspect of the present invention, a method is provided to identify waterborne biological organisms, wherein said probe comprises a nucleotide sequence designated SEQ ID NO. O, wherein O is an integer selected from the group consisting of each integer in the bounded interval 13 to 16, inclusive.

According to an aspect of the present invention, a method is provided to identify waterborne biological organisms, according to claim 1, wherein said primer comprises a nucleotide sequence designated SEQ ID NO. N, wherein N is an integer selected from the group consisting of each integer in the bounded interval 1 to 12, inclusive.

According to an aspect of the present invention, at least one kit is provided suitable for use in an automated PCR-amplified DNA detection system said at least one kit comprising: a multi-well plate; control DNA; primers; and probes.

According to an aspect of the present invention, at least one kit is provided suitable for use in an automated PCR-amplified DNA detection system, wherein said primer is an oligonucleotide selected from the group consisting of SEQ ID NO.: N, where N is an integer in the bounded interval 1-12, inclusive.

According to an aspect of the present invention, at least one kit is provided suitable for use in an automated PCR-amplified DNA detection system wherein said probe is an oligonucleotide selected from the group consisting of SEQ ID NO.: O, where O is an integer in the bounded interval 13-16, inclusive.

According to an aspect of the present invention, at least one kit is provided suitable for use in an automated PCR-amplified DNA detection system wherein said control DNA is an isolated and purified oligodeoxynucleotide obtained from a water borne organism wherein said organism is selected from the group consisting of Pfiesteria, Aureococcus, Gymnodinium, Chattonella, and Alexandrium.

According to an aspect of the present invention, a method is provided to identify waterborne biological organisms, wherein said primer co rises a nucleotide sequence designated SEQ ID NO. N, wherein N is an integer selected from t group consisting of each integer in the bounded interval 1 to 12, inclusive.

According to an aspect of the present invention, a method is provided to identify waterborne biological organisms, wherein said probe comprises a nucleotide sequence designated SEQ ID NO. O, wherein O is an integer selected from the group consisting of each integer in the bounded interval 11 to 16, inclusive.

According to an aspect of the present invention, the invention comprises a nucleic acid primer for amplifying DNA comprising an oligonucleotide sequence selected from the group consisting of SEQ ID NO.: N, wherein N is an integer selected from the group consisting of each integer in the bounded interval 1 to 12, inclusive.

According to an aspect of the present invention, the invention comprises a nucleic acid probe for detecting and quantifying amplicons comprising an oligonucleotide sequence selected from the group consisting of SEQ ID NO.: O, wherein O is an integer selected from the group consisting of each integer in the bounded interval 13 to 16, inclusive.

Still other objects and advantages of the present invention will become readily apparent by those skilled in the art from the following detailed description, wherein it is shown and described preferred embodiments of the invention, simply by way of illustration of the best mode contemplated of carrying out the invention. As will be realized the invention is capable of other and different embodiments, and its several details are capable of modifications in various obvious respects, without departing from the invention. Accordingly, the description is to be regarded as illustrative in nature and not as restrictive.

### DETAILED DESCRIPTION OF A PREFERRED EMBODIMENT

The present invention relates generally to methods to amplify and detect DNA using PCR methods. Those skilled in the arts will be familiar with the teachings of PCR as set forth in standard reference works such as for example, Innis et al., PCR Protocols, A Guide to Methods and Applications (Academic Press, Inc.; 1990)(“Innis”), Sambrook et al. (1989) Molecular Cloning, A Laboratory Manual (2d Edition), Cold Spring Harbor Press, Cold Spring Harbor, N.Y. (“Sambrook”), and Ausubel et al., eds. (1996) Current Protocols in Molecular Biology, Current Protocols, a joint venture between Greene Publishing Associates, Inc. and John Wiley & Sons, Inc. (“Ausubel”). Moreover, those skilled in the art will be familiar with the PCR teachings of Mullis U.S. Pat. Nos. 4,683,195, No. 4,683,202, and 4,965,188). PCR is an in vitro DNA amplification procedure which requires two primers that flank the target sequence to be synthesized. A primer is an oligonucleotide sequence which is capable of hybridizing in a sequence specific fashion to the target sequence and extending during the PCR reaction. Amplicons, PCR products, and PCR fragments are extension products which comprise the primer and the newly synthesized copies of the target sequences

Reagents. Reagents for the practice of PCR and related reactions are amply described in the prior art. For example, Innis, Sambrook, and Ausubel, all supra, each provide substantial teaching regarding optimal reagents for PCR. In addition, numerous references on the Internet provide protocols and methods for PCR and related reactions (see, for example, www.alkami.com/or www.promega.com/amplification/prodguide/).

Serial Filtration. A water sample is obtained, either as an environmental sampling or preferably, as a sample of ballast water from, for example, a cargo ship. The water sample is divided into a plurality of aliquots. Typically the size of an aliquot is about 1000 milliliters and preferably is about 5000 milliliters although any convenient size may be used. Each aliquot is separately filtered through a first filter having a pore size corresponding to the size of the largest organism that is anticipated might be found in the sample. By way of non-limiting example, a first filter having a 20 micrometer pore might be used to trap Chattonella species. The process of filtration divides the aliquot into a first retentate, material retained on and in the first filter, and a first filtrate, the liquid volume and solid material present in the liquid volume that passed through the first filter.

The first filtrate is subjected to a second filtration using a filter having a pore size smaller that the pore size of the first filter. By way of non-limiting example, a second filter having a 5 micrometer pore might be used to trap Pfiesteria species. The second filtrate may be subjected to a third filtration. By way of non-limiting example, a third filter having a 1 micrometer pore might be used to trap species associated with brown tide.

It is understood that a preferred embodiment comprises serial filtration. However, the invention is compatible with a single filtration, provided that pore size is small enough to trap the species of interest. Those skilled in the art will readily comprehend how to chose an appropriate pore Size.

Each filter, along with its associated retentate, is placed in a tube containing 0.5 mL CTAB buffer (CTAB tube) which was previously heated to, and maintained at, about 50° C. CTAB buffer comprises about 1.4 molar NaCl, about 20 millimolar ethylenediaminetetraacetic acid (EDT A), about 0.4% (vol/vol) mercaptoethanol (preferably added immediately prior to use), about 2% (wt/vol) cetyltrimethylammonium bromide (CTAB), about 1% (wt/vol) polyvinylpyrolidone (PVP), and about 100 millimolar trishydroxymethylaminomethane (Tris) adjusted to a pH of about 8.0 with HCl. It is preferred that the PVP have an average molecular,-:eight of about 360,000. After a filter, and associated retatate, is placed in a CTAB tube, the retentate may be processed to extract DNA immediately, or tubes containing retentate may be stored at −80° C. for later processing. Those skilled in the art will understand that suitable cationic detergents may be substituted for CTAB.

Template DNA Extraction. DNA may be recovered from the retentate by any of a variety of methods known to the art. Methods for the preparation of templates can be found in a multitude of sources, including Innis, Sambrook, Ausubel, all supra. Any such method can be used in the present invention. Typically, these methods involve cell lysis, followed by purification of nucleic acids by methods such as phenol/chloroform extraction, electrophoresis, and/or chromatography. Often, such methods include a step wherein the nucleic acids are precipitated, for example, with ethanol, and resuspended in an appropriate buffer for addition to a PCR or similar reaction. In a preferred method, CT AB tubes, containing filters and associated retentates, are incubated for about 15 minutes at about 65° C. The tubes are allowed to cool briefly and an equal volume (about 0.5 milliliters) of a mixture of chloroform and isoamyl alcohol (24/1, vol/vol) is added. The mixture is rocked on a rocker for about 20 minutes. The liquid is separated into an aqueous and an organic phase by centrifugation at about 17,000 relative centrifugal force for 15 minutes. The aqueous phase is quantitatively removed to a fresh tube and about 0.5 volumes NaCl (about 5 molar concentration) and 1 volume isopropanol are added. Incubate at −80° C. for at least 1 hour. Pellet the DNA by centrifugation at about 17,000 relative centrifugal force for about 30 minutes. Wash pellet with 0.5 milliliter of ethanol (70% vol/vol). Dry pellet and suspend it in about 10 to about 50 microliters of sterile, deionized water, preferably water having been treated to remove any organic or ionic contaminants. Thus an aspect of the present invention provides DNA isolated from organisms present in the ballast water of a ship wherein the DNA is segregated according to the size of the organism from which it was isolated.

Template. As used herein, the term “template” refers to any amplifiable nucleic acid molecule. Any source of nucleic acid, in purified or non purified form, can be utilized as the starting nucleic acid or acids, provided it contain, or is suspected of containing, the specific nucleic acid sequence desired. Thus, the process may employ, for example, DNA or RNA, including messenger RNA, which DNA or RNA may be single stranded or double stranded. In addition, a DNA-RNA hybrid which contains one strand of each may be utilized. A mixture of any of these nucleic acids may also be employed, or the nucleic acid produced from a previous amplification reaction herein using the same or different primers may be so utilized. It is not necessary that the sequence to be amplified be present initially in a pure form; it may be a minor fraction of a complex mixture, such as a portion of the gene contained in whole human DNA or a portion of nucleic acid sequence due to a particular microorganism which organism might constitute only a very minor fraction of a particular biological sample. The starting nucleic acid may contain more than one desired specific nucleic acid sequence which may be the same or different. As a non-limiting example, a “sample template” may be DNA, isolated from an organism obtained from the filtrate of an aliquot of water derived from the ballast of a ship or from an environmental sample. As a non-limiting example, a “control template” may be DNA isolated from a known organism which may be obtained, for example, from the American Type Culture Collection (Manassas, Va.). A “blank template” may be a volume of water, possibly containing salts, buffers, and other materials, but substantially free of amplifiable nucleic acid molecules. A “blank template” serves as a negative control. Where the template comprises DNA, the DNA may be of any convenient size. The templates may be from about 20-50, 50 100, 100-150, 150-175, 175-300,300-500,500-1000 nucleotides or longer. Template DNA or RNA may be extracted from any tissue material by any of a variety of techniques such as that described by Maniatis et al., Molecular Cloning A Laboratory Manual (New York: Cold Spring Harbor Laboratory, 1982).

High Throughput, Real-Time PCR. An aspect of the present invention provides for the specific amplification and detection of DNA wherein the specificity is provided by targeting predetermined organisms. A further aspect of the present invention provides the ability to target any organism that may be present in environmental or ballast water samples.

DNA, extracted from environmental or ballast water samples, is amplified by PCR processes known in the art. Non-limiting examples of PCR protocols are set forth in standard reference works such as Innis, Stambrook, and Ausubel supra. The Internet provides numerous references to protocols, automated devices, reagents, and methods for PCR and related reactions (see, for example, www.alkami.com/or www.promega.com/amplification/prodguide/). In a preferred embodiment, PCR amplification is performed using an automated system providing real-time detection of PCR-amplified DNA.

In a more preferred embodiment, the automated PCR system is an ABI Prism™ 7700 Sequence Detection System (Applied Biosystems). The ABI PRISM™ 7700 Sequence Detection System is a fully integrated system for real-time detection of PCR. The system includes a built-in thermal cycler, a laser to induce the dyes used to detect and quantify the amplicons to fluoresce, a CCD (charge-coupled device) detector, real-time sequence detection software, and TaqMan™ reagents for the fluorogenic 5′ nuclease assay. Reagents and instructions for use with the 7700 system may be obtained from Applied Biosystems. Persons skilled in the arts will understand that the invention is capable of being performed using other automatic, or semi-automatic, systems.

The amplification and detection reactions are carried out in multi-well plates adapted for the automated system chosen. By way of non-limiting example, 96-well plates may be used. The individual wells of the plates are prepared to contain primers and probes specific to the genus and species of organism that is desired to be detected.

Primer. The term “primer” as used herein refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, i.e., in the presence of nucleotides and an inducing agent such as DNA polymerase and at a suitable temperature and pH. The primer is preferably single stranded for maximum amplification efficiency, but, in the alternative, may be double stranded. If double stranded, the primer is first treated to separate its strands before being used to prepare extension products. Preferably, the primer is an oligodeoxyribonucleotide. The oligonucleotide primers can be DNA, RNA, chimerical mixture, or derivatives or modified versions thereof, so long as it is still capable of priming the desired reaction. The oligonucleotide primer can be modified at the base moiety, sugar moiety, or phosphate backbone, and may include other appending groups or labels, so long as it is still capable of priming the desired amplification reaction The primer must be sufficiently long to prime the synthesis of extension products in the presence of the inducing agent. The exact lengths of the primers will depend on many factors, including temperature, source of primer and use of the method. For example, for diagnostics applications, depending on the complexity of the target sequence, the oligonucleotide primer typically contains 15-25 or more nucleotides, although it may contain fewer nucleotides. For other applications, the oligonucleotide primer is typically shorter, e.g., 7-15 nucleotides. Such short primer molecules generally require cooler temperatures to form sufficiently stable hybrid complexes with template. The primers herein are selected to be “substantially” complementary to the different strands of each specific sequence to be amplified. This means that the primers must be sufficiently complementary to hybridize with their respective strands.

Oligonucleotides for use as primers are typically synthesized chemically according to the solid phase phosphoramidite triester method described by Beaucage and Caruthers (1981), Tetrahedron Letts., 22(20):1859-1862, using an automated synthesizer, as described in Needham- VanDevanter et al. (1984) Nucleic Acids Res., 12:6159-6168. Oligonucleotides can also be custom made and ordered from a variety of commercial sources known to persons of skill. Purification of oligonucleotides, where necessary, is typically performed by either native acrylamide gel electrophoresis or by anion-exchange HPLC as described in Pearson and Regnier (1983) J. Chrom. 255:137-149. The sequence of the synthetic oligonucleotides can be verified by any of several methods. Non-limiting examples include the chemical degradation method of Maxam and Gilbert (1980) as described in Methods in Enzymology 65:499-560 Grossman and Moldave (eds.) Academic Press, New York.

Although primers can hybridize to any of a number of sequences, selecting optimal primers is typically done using computer assisted consideration of available sequences and excluding potential primers which do not have desired hybridization characteristics, and/or including potential primers which meet selected hybridization characteristics. This is done by determining all possible nucleic acid primers, or a subset of all possible primers with selected hybridization properties (e.g., those with a selected length, G:C ratio, uniqueness in the given sequence, etc.) based upon the known sequence. The selection of the hybridization properties of the primer is dependent on the desired hybridization and discrimination properties of the primer. In general, the longer the primer, the higher the melting temperature. As noted above, any desired primer can be synthesized using standard methods. In general, it is expected that one of skill is thoroughly familiar with the theory and practice of nucleic acid hybridization and primer selection. Gait, ed. Oligonucleotide Synthesis: A Practical Approach, IRL Press, Oxford (1984); W. H. A. Kuijpers Nucleic Acids Research 18(17),5197 (1994); K. L. Dueholm J. Org. Chem. 59,5767-5773 (1994); S. Agrawal (ed.) Methods in Molecular Biology, volume 20; and Tijssen (1993) Laboratory Techniques Biochemistry and Molecular Biology--Hybridization with Nucleicleic Acid Probes, e.g., part I, Chapter 2 “Overview of Principles of Hybridization and the Strategy of Nucleic Acid Probe Assays”, Elsevier, N. Y. provides a basic guide to nucleic acid hybridization. Innis supra provides an overview of primer selection.

One of skill will recognize that there are a variety of possible ways of performing the primer selection steps, and that variations on the steps are appropriate. Most typically, selection steps are performed using simple computer programs to perform the selection as outlined above; however, all of the steps are optionally performed manually. One available computer program for primer selection is the Mac Vector program from Kodak. An alternate program is the MFOLD program (Genetics Computer Group, Madison Wis.) that predicts secondary structure of, e.g., single-stranded nucleic acids. In addition to programs for primer selection, one of skill can easily design simple programs for any or all of the preferred selection steps.

An embodiment of the present invention provides primers that hybridize under stringent conditions to nucleotide sequences specific to Pfiesteria piscicida. A preferred primer of the present invention, for example, comprises a sequence selected from the group consisting of: SEQ ID NO.: 1 and SEQ ID NO.: 2.

An embodiment of the present invention provides primers that hybridize under stringent conditions to nucleotide sequences specific to Pfiesteria shumwayae. A preferred primer of the present invention, for example, comprises a sequence selected from the group consisting of: SEQ ID NO.: 3 and SEQ ID NO.: 4.

An embodiment of the present invention provides primers that hybridize under stringent conditions to nucleotide sequences specific to Aureococcus anophagefferens. A preferred primer of the present invention, for example, comprises a sequence selected from the group consisting of: SEQ ID NO.: 6, SEQ ID NO.: 7, SEQ ID NO.: 8 and SEQ ID NO.: 9.

An embodiment of the present invention provides primers that hybridize under stringent conditions to nucleotide sequences specific to Chattonella. A preferred primer of the present invention, for example, comprises a sequence selected from the group consisting of: SEQ ID NO.: 10, SEQ ID NO.: 11 and SEQ ID NO.: 12.

Persons skilled in the art will understand the term “stringent” refers to conditions under which a first oligonucleotide hybridizes to a second oligonucleotide only where said first and said second oligonucleotides are highly complementary. Preferably, the hybridization medium will contain standard buffers and detergents. More preferably, the hybridization medium contains a buffer such as Tris-HCl, PIPES or HEPES, preferably Tris-HCl at a concentration of about 0.05 to 0.5 Molar. The hybridization medium will preferably also contain about 0.05 to 0.5% (wt./vol.) of an ionic or non-ionic detergent, such as sodium dodecylsulphate (SDS) or Sarkosyl (Sigma Chemical Co., St. Louis, Mo.) and between 1 and 10 mM EDTA. Other additives may also be included, such as volume exclusion agents (chaotropes) which include a variety of polar water-soluble or swellable agents, such as anionic polyacrylate or polymethacrylate, and charged saccharide polymers, such as dextran sulphate and the like. The specificity or the stringency of hybridization may be controlled, for instance, by varying the concentration and type of chaotropic agent and the NaCl concentration which is typically between 0 and 1M. Typically, the NaCl concentration is between about 0.1 and about 1.0M. Increasing the NaCl concentration increases the stringency. The degree of complementarity required for formation of a stable hybridization complex (duplex) varies with the stringency of the hybridization medium and/or wash medium. Primers and probes and methods for making and labeling them are described in Andrus, A. “Chemical methods for 5′ non-isotopic labeling of PCR probes and primers” (1995) in PCR 2: A Practical Approach, Oxford University Press, Oxford. As is known to persons skilled in the art, a forward primer is used with a reverse primer.

Amplicon. As used herein, the term “amplicon” refers to the DNA molecule, or molecules, produced by the PCR reaction. It is understood that an amplicon may be a single-stranded DNA molecule. It is understood that an amplicon may be a double-stranded DNA molecule. The present invention is directed to a process for amplifying any one or more desired specific nucleic acid sequences found in a target nucleic acid. The large amounts of the specific sequence(s) be produced by this process are termed “amplicon.” In the context of the invention, “quantitatively detecting” or “quantifying” refers to a process for determining the extent of polynucleotide or amplicon production.

Probe. As used herein, the term “probe” refers to an oligonucleotide molecule capable of hybridizing to an amplicon molecule under stringent conditions. It is understood that probes provide means of detecting amplicon molecules. A “labeled probe” is a polynucleotide that harbors a detectable moiety and that can combine with a complementary single-stranded target nucleic acid sequence to form a double-stranded hybrid. The term also includes analogs of naturally occurring nucleotides and particularly includes analogs having a methoxy group (OMe) at the 2′ position of the ribose. The detectable moiety may be attached to the end(s) of the probe or may be positioned internally within the sequence of the probe. In general, labeled probes will be about 10 to about 100 nucleotides in length, but can be longer than 100 or shorter than 10 nucleotides. A “detectable moiety” is a molecule attached to, or synthesized as part of, a labeled probe. This molecule should be uniquely detectable and will allow the probe to be detected as a result. These detectable moieties are often radioisotopes, e.g, ³²p, ³H, 35s, ¹⁴C; chemiluminescent molecules, e.g, the intercalating dye SYBR Green™ (Molecular probes Inc.), TAQ MAN™ probe (Roche Molecular Systems); enzymes; haptens; or even unique oligonucleotide sequences. Probes may provide means of detection without themselves being labeled. For example there are so called “intercalating” dyes that do not bind to single-stranded oligonucleotide molecules, but bind strongly to double-stranded oligonucleotide molecules. Intercalating dyes are known in the art (e.g., SYBR Green™ (Molecular probes Inc.)) as means of detecting the presence of amplicon-probe duplexes.

An embodiment of the present invention provides probes that hybridize under stringent conditions to nucleotide sequences specific to P. piscicida. In a preferred embodiment of the present invention the probe comprises SEQ ID NO.: 13.

An embodiment of the present invention provides probes that hybridize under stringent conditions to nucleotide sequences specific to P. shumwayae. In a preferred embodiment of the present invention the probe comprises SEQ ID NO.: 14.

An embodiment of the present invention provides probes that hybridize under stringent conditions to nucleotide sequences specific to A. anophagefferens. A preferred probe of the present invention, for example, comprises a sequence selected from the group consisting of: SEQ ID NO.: 15 and SEQ ID NO.: 16.

An embodiment of the present invention provides microarray methods for hybridizing target DNA with the appropriate primers and probes. In an embodiment of the present invention, the PCR reactions are carried out in microarray formats. The term “solid support” refers to any surface onto which targets, such as nucleic acids, may be immobilized for conducting assays and reactions.

The term “target”, “DNA element” or “microarray element” refers to a molecule that has an affinity for a given sample. Targets may be naturally occurring or synthetic molecules, and may be attached, covalently or noncovalently, to a surface, either directly or via a specific binding substance. Examples of targets which can be employed by this invention include, but are not restricted to, DNA, RNA, oligonucleotides, oligosaccharides, polysaccharides, sugars, proteins, peptides, PNAs, monoclonal antibodies, toxins, viral epitopes, hormones, hormone receptors, enzymes, enzyme substrates, cofactors, and drugs including agonists and antagonists for cell surface receptors.

The term “microarray” refers to an array of targets synthesized or attached or deposited on a substrate, such as paper, nylon or other type of membrane, filter, chip, glass slide, beads, or any other suitable solid support, at high density.

In one embodiment, polynucleotides and activated polynucleotides are attached to an amine treated solid support, such as a glass slide. The attachment can be covalent, non-covalent, or a combination of the two. The array or the microarray thus manufactured may be contacted, under hybridization conditions, with a fluid sample containing labeled nucleic acid probes complementary to the polynucleotide target. The hybridization pattern from the probe may be detected to obtain information about the genetic profile of the labeled nucleic acid sample.

Persons of skill will recognize that the arrays may be produced according to any convenient methodology, and a number of different array configurations and methods for their production are known to those of skill in the art and disclosed in U.S. Pat. Nos.: 5,445,934, 5,532,128; 5,384,261; and 5,700,637; the disclosure of which are herein incorporated in their entirety by reference. For example, the polynucleotides may be stably associated with the substrate through non-covalent means, such as by interacting with a positively charged surface of the substrate. Alternatively, the polynucleotides possessing the 5′- or the 3′-terminal linker arm may be bound to the substrate through covalent means, such as via an alkylamino-linker group. In another embodiment of the invention, the polynucleotides may be associated with the substrate through both non-covalent and covalent means.

In order to conduct sample analysis using the arrays and microarrays of the invention, the RNA or DNA from a biological sample is made into hybridization probes, as is well known in the art. The hybridization probes are then contacted with the arrays and microarrays of the invention under hybridization conditions to produce a hybridization pattern. Suitable hybridization conditions are well known to those of skill in the art and reviewed in WO 95/21944 to Maniatis et al. Incubation conditions are adjusted so that hybridization occurs with precise complementary matches or with various degrees of less complementarity. After removal of nonhybridized probes, a scanner is used for detection or visualization to determine the levels and patterns of fluorescence. A detection system may be used to measure the absence, presence, and amount of hybridization for all of the distinct sequences simultaneously.

### EXAMPLE 1

### Identification of Dinoflagellate Species

A typical embodiment of the present invention suitable for the detection and characterization of dinoflagellate species employs a 96-well plate having eight rows of twelve columns. Persons skilled in the art will recognize that other multiwell formats may be used.

Typically, the first two columns comprise replicate negative control wells to which no template DNA is added.

Typically third and fourth columns comprise duplicate positive control wells to which is added a plasmid or other source of known DNA.

The remaining columns comprise test wells to which samples of DNA extracted from ballast water or other environmental samples is added. Wells of each row contain primers and probes specific to a target. The first row, to the wells of which is added universal primers and probe specific to the 185 rRNA gene, tests for the presence of eukaryotic organisms. A universal primer set comprises oligonucleotide primers that are designed to amplify a specific segment of DNA from the 185 rDNA gene of all eukaryotic organisms. These primers are designed from highly conserved sequences in eukaryote 185 rDNA genes which may be identified by comparison of gene sequences found in public domain databases such as GenBank. A universal probe comprises an oligonucleotide sequence found within the segment of DNA amplified using the universal primer set. The probe is designed from highly conserved sequences in eukaryote 185 rDNA genes which may be identified by comparison of gene sequences found in public domain databases such as GenBank.

A second row typically comprises wells for the identification of the presence of dinoflagellates (Dinophyceae). To these wells are added primers and probes specific to dinoflagellates. Preferred dinoflagellate-specific primer sets include oligonucleotide primers that are designed to amplify a specific segment of DNA from the 185 rDNA gene of all dinoflagellate species. These primers are designed from conserved sequences in Dinophyceae 185 rDNA genes which may be identified by comparison of gene sequences found in public domain databases such as GenBank. Preferred dinoflagellate-specific probes include an oligonucleotide sequence found within the segment of DNA amplified using the dinoflagellate primer set. The probe is designed from conserved sequences in dinoflagellate 185 rDNA genes which may be identified by comparison of gene sequences found in public domain databases such as GenBank. A third row typically comprises wells for the identification of the specific genus, nonlimiting examples of which include Pfiesteria and Gymnodinium. Preferred Pfiesteria-specific primer sets include oligonucleotide primers that are designed to amplify a specific segment of DNA from the 185 rDNA gene of all Pfiesteria or Gymnodinium species. These primers are designed from conserved sequences in Pfiesteria or Gymnodinium 185 rDNA genes which may be identified by comparison of gene sequences found in public domain databases such as GenBank. By way of non-limiting example, AF363589 refers to Pgiesteria spp. clone 8A 185 ribosomal RNA gene, partial sequence on deposit at GenBank as GI: 13937048; AF363588 refers to Pfiesteria spp. clone 4B 185 ribosomal RNA gene, partial sequence on deposit at GenBank as GI: 13937047; AF363587 refers to Pfiesteria spp. clone 20B 18S ribosomal RNA gene, partial sequence on deposit at GenBank as GI: 13937046; AF363586 refers to Pfiesteria spp. clone 17B 18S ribosomal RNA gene, partial sequence on deposit at GenBank as GI: 13937045; and AF363585 refers to Pflesteria spp. clone 14B 18S ribosomal RNA gene, partial sequence on deposit at GenBank as GI: 13937044. The sequences for each of these was deposited by the present inventors and are specifically incorporated by reference, in their entirety and for all purposes.

Preferred Pfiesteria-specific probes include an oligonucleotide sequence found within the segment of DNA amplified using the Pfiesteria primer set. The probe is designed from conserved sequences in Pfiesteria 18S rDNA genes which may be identified by comparison of gene sequences found in public domain databases such as GenBank. Preferred Gymnodinium-specific probes include an oligonucleotide sequence found within the segment of DNA amplified using the Gymnodinium primer set. The probe is designed from conserved sequences in Gymnodinium 18S rDNA genes which may be identified by comparison of gene sequences found in public domain databases such as GenBank.

Subsequent rows typically comprise wells for the detection and identification of individual species within a genus. By way of non-limiting example, the genus Pfiesteria includes P. piscicida, and P. shumwayae. Preferred P. piscicida-specific primers include Pp156F and Pp290R, (See Table I). Preferred P. piscicida-specific probes include Pp215, (See Table II). Preferred P. shumwayae-specific primers include Ps56F and Ps299R, (See Table I). Preferred P. shumwayae-specific probes include Pshum, (See Table II).

TABLE I

SEQ

| | | | |
|---|---|---|---|
| Organism | ID | Name | Primer Sequence |

| | | | |
|---|---|---|---|
| Pfisteria piscicida | 1 | Pp156F | TAGAGCTAATACATGCACCAAAGC |
| P. piscicida | 2 | Pp290R | TGATAGGTCAGAAAGTGATATGGTATAT |
| P. shumwayae | 3 | Ps56F | TGTCTCAGTTTAAGTCCCAGT |
| P. shumwayae | 4 | Ps299R | AGCTGATAGGTCAGAATCTGAT |
| Aureococcus | 5 | AanoF-3 | CAAAAGGTGCGCACTGAGCG |
| anophagefferens | | | |
| A. anophagefferens | 6 | AanoR | CGACAACTGAATGCCAGGATAGAC |
| A. anophagefferens | 7 | Aa631F | GTAGTTGGATTCCTGGCAC |
| A. anophagefferens | 8 | Aa607F | CCATCCTTGCGATGGTCTA |
| A. anophagefferens | 9 | Aa1685F | ACCTCCGGACTGGGGTT |
| Chattonella | 10 | Raph1350F | GCTAAATAGTGTsGGTAATGCTT |
| Raphidophyte spp. | | | |
| Chattonella | 11 | Raph1705R | GGCAAGTCACAATAAAGTTCCA |
| Chattonella | 12 | Raph1370R | TAAGAAGTAGAAAmGGCAATGC |

One skilled in the art will understand that an “F” in the designation of a primer refers to a forward primer, whereas an “R” refers to a reverse primer. Primer designations are given in Table I, Column 3 (Name). Moreover, a person skilled in the art will understand that an “s” in the listed DNA sequence refers to a degenerate position in which either a “G” or “C” may be substituted in the nucleic acid sequence. Furthermore, a persons skilled in the art will understand that an “m” in the listed DNA sequence refers to a degenerate position in which either a “A” or “C” may be substituted in the nucleic acid sequence.

TABLE II

SEQ

| | | | |
|---|---|---|---|
| Organism | ID | Name | Sequence |

| | | | |
|---|---|---|---|
| P. piscicida | 13 | Pp215 | ACAGAACCAACCCAGGCTTGCCTG |
| P. shumwayae | 14 | Pshum | TACATGCACCCAAGCCCGACTCC |
| A. anophagefferens | 15 | Aa694 | CGGCCTCGGCCATCCTTGCG |
| A. anophagefferens | 16 | Aanoprobe 1714 | CCAACTTCCCGAGGTACGTTCGCC |

Kits. Generally, the reagents and devices described herein are packaged to include at least a portion of the necessary components for performing the reactions described herein. For example, the kits can include any of templates, polymerases, primers, probes buffers and other chemical agents, nucleotides, sample materials, control materials, devices, or the like. Such kits also typically include appropriate instructions for using the devices and/or reagents, and in cases where reagents are not predisposed in the devices themselves, with appropriate instructions for introducing the reagents into the devices. Another aspect of the invention provides a kit suitable for the identification of water-borne species. The kit of the present invention comprises control DNA and a multi-well plate to which primers and probes have been added. The positive control DNA is included in separate vials. The primers and probes is lyophilized in the wells of the 96-well plate. Plates of the present inventive kit are typically stored at −20° C.

The foregoing description of the invention illustrates and describes the present invention. Additionally, the disclosure shows and describes only the preferred embodiments of the invention but, as mentioned above, it is to be understood that the invention is capable of use in various other combinations, modifications, and environments and is capable of changes or modifications within the scope of the inventive concept as expressed herein, commensurate with the above teachings and/or the skill or knowledge of the relevant art. The embodiments described hereinabove are further intended to explain best modes known of practicing the invention and to enable others skilled in the art to utilize the invention in such, or other, embodiments and with the various modifications required by the particular applications or uses of the invention. Accordingly, the description is not intended to limit the invention to the form disclosed herein. Also, it is intended that the appended claims be construed to include alternative embodiments.

Incorporation by reference. All patent documents and publications, specifically including nucleotide sequences published at GenBank, cited in this application are incorporated by reference in their entirety for all purposes to the same extent as if each individual publication or patent document were so individually denoted. Moreover, the present application specifically incorporates by reference in its entirety and for all purposes the manuscript submitted for publication as Coyne, K., Hutchins, D. A., Hare, C., and Cary, C., Temporal and Spatial Variability in Pfiesteria piscicida Distributions Using Molecular Probing Techniques, to be published as 24 Aquatic Microbial Ecology 275 (2001).

What we claim is:

1. A method to identify water-borne organisms associated with harmful algal bloom the method comprising the steps of:
providing an aliquot of water, possibly containing biological organisms;
fractionating said aliquot by size to yield a plurality of fractions;
isolating DNA from at least one of said fractions;
amplifying said DNA through the process of high throughput, real-time PCR, wherein said PCR comprises at least one forward genus-specific primer and at least one reverse genus-specific primer to yield an amplified DNA amplicon;
contacting said amplicon with at least one species-specific labeled probe; and
detecting said amplicon.

2. A method to identify water-borne organisms associated with harmful algal bloom, according to claim 1, wherein said fractioning comprises serial filtration.

3. A method to identify water-borne organisms associated with harmful algal bloom, according to claim 1, wherein said organisms are dinoflagellates selected from the group consisting of Pfiesteria, Gymnodinium, Chattonella, Alexandrium, and Aureococcus.

4. A method to identify water-borne organisms associated with harmful algal bloom, according to claim 1, wherein said organisms are transported in the ballast of a ship.

5. A method to identify water-borne organisms transported in the ballast of a ship, said method comprising the steps of:
providing an aliquot of ship ballast water, possibly containing biological organisms;
fractionating said aliquot by size to yield a plurality of fractions;
isolating DNA from at least one of said fractions;
amplifying said DNA through the process of high throughput, real-time PCR, wherein said PCR comprises at least one forward genus-specific primer and at least one reverse genus-specific primer to yield an amplified DNA amplicon;
contacting said amplicon with at least one species-specific labeled probe; and
detecting said amplicon.

6. A method to identify invasive organisms transported in the ballast of a ship, according to claim-5, wherein said organism is a zebra mussel (Dreissena).

7. A method to identify water-borne biological organisms, according to claim 2 wherein said serial filtration comprises the steps of:
filtering said aliquot through at least a first filtration means having a first pore size, thereby obtaining a first filtrate and a first retentate; and
filtering said first filtrate through at least a second filtration means wherein said second filtration means has a second pore size smaller than said first pore size, thereby obtaining at least a second filtrate and a second retentate.

8. A method to identify water-borne biological organisms, according to claim 2, wherein
said aliquot comprises ship ballast water.

9. A method to identify water-borne biological organisms, according to claim 2, wherein said aliquot comprises an environmental sampling.

10. A method to identify water-borne biological organisms, according to claim 2, wherein isolating DNA comprises the steps of:
extracting said retentate with an organic solvent thereby forming an aqueous fraction and an organic fraction; and
extracting DNA from said aqueous fraction thereby forming test DNA.

11. A method to identify water-borne biological organisms, according to claim 2, wherein
amplifying said DNA employs at least one forward primer and at least one reverse primer.

12. A method to identify water-borne biological organisms, according to claim 1, wherein high throughput real-time PCR analysis comprises the steps of:
(a) providing an automated system for detection of amplified DNA sequences;
(b) providing a charged plate compatible with said system, wherein said plate comprises a plurality of wells;
(c) providing said extracted DNA to said charged plate;
(d) amplifying said DNA; and
(e) detecting said amplified DNA.

13. A method to identify water-borne organisms associated with harmful algal bloom, according to claim 12, wherein said automated DNA detection system comprises operating instructions.

14. A method to identify water-borne biological organisms, according to claim 12; wherein said plate comprises:
at least one well established as a negative control;
at least one well established as a positive control;
at least one well provided test DNA;
a plurality of wells containing sample DNA;
appropriate primers; and
appropriate probes.

15. A method to identify water-borne biological organisms, according to claim 1, wherein detecting said DNA amplicon comprises hybridizing said PCR-amplified DNA amplicon with a labeled probe.

16. A method to identify water-borne biological organisms, according to claim 1, wherein said probe comprises a nucleotide sequence designated SEQ ID NO. O, wherein O is an integer selected from the group consisting of each integer in the bounded interval 13 to 16 , inclusive.

17. A method to identify water-borne biological organisms, according to claim 1, wherein said primer comprises a nucleotide sequence designated SEQ ID NO. N, wherein N is an integer selected from the group consisting of each integer in the bounded interval 1 to 12, inclusive.

18. A kit suitable for use in an automated PCR-amplified DNA detection system according to claim 1 comprising:
a multi-well plate;
control DNA;
primers; and
probes.

19. A kit suitable for use in an automated PCR-amplified DNA detection system according to claim 13, wherein said primer is an oligonucleotide selected from the group consisting of SEQ ID NO.: N, where N is an integer in the bounded interval 1-12, inclusive.

20. A kit suitable for use in an automated PCR-amplified DNA detection system according to claim 13, wherein said probe is an oligonucleotide selected from the group consisting of SEQ ID NO.: O, where O is an integer in the bounded interval 13-16, inclusive.

21. A kit suitable for use in an automated PCR-amplified DNA detection system according to claim 13, wherein said control DNA is an isolated and purified oligodeoxynucleotide obtained from a water borne organism wherein said organism is selected from the group consisting of Pfiesteria, Aureococcus, Gymnodinium, Chattonella, and Alexandrium.

22. A method to identify water-borne biological organisms, according to claim 1, wherein said primer comprises a nucleotide sequence designated SEQ ID NO. N, wherein N is an integer selected from the group consisting of each integer in the bounded interval 1 to 12, inclusive.

23. A method to identify water-borne biological organisms, according to claim 1, wherein said probe comprises a nucleotide sequence designated SEQ ID NO. O, wherein O is an integer selected from the group consisting of each integer in the bounded interval 11 to 16, inclusive.

24. A primer for amplifying DNA comprising an oligonucleotide selected from the group consisting of SEQ ID NO.: N, wherein N is an integer selected from the group consisting of each integer in the bounded interval 1 to 12, inclusive.

25. A probe for detecting and quantifying amplicons comprising an oligonucleotide selected from the group consisting of SEQ ID NO.: O, wherein O is an integer selected from the group consisting of each integer in the bounded interval 13 to 16, inclusive.

26. A method to identify water-borne biological organisms, according to claim 1, wherein high throughput real-time PCR analysis comprises microarrays.
